(19) United States
(12) Patent Application Publication
(10) Pub. No.: US 2006/0005844 A1
(43) Pub. Date: Jan. 12, 2006

(54) Rolling tube apparatus and method for treating a wound

(76) Inventor: George Gary Coulter, Nashville, IN (US)

Correspondence Address: WILSON SONSINI GOODRICH & ROSATI, 650 PAGE MILL ROAD, PALO ALTO, CA, 94304-1050, US

(21) Appl. No.: 11/178,249

(22) Filed: Jul. 8, 2005

Related U.S. Application Data
(60) Provisional application No. 60/586,447, filed on Jul. 8, 2004.

(51) Int. Cl. A61B 17/00

(52) U.S. Cl. 128/887

(57) ABSTRACT

An apparatus inhibits hemorrhaging of a wound of a patient. A casing includes a nozzle to be inserted into the wound. A torus-shaped flexible element is disposed in the casing and defines a longitudinal direction. An actuating device moves the flexible element in the longitudinal direction through the nozzle and into the wound.

### CROSS-REFERENCE

This application claims the benefit of U.S. Provisional Application No. 60/586,447, filed Jul. 8, 2004, which is incorporated herein by reference in its entirety.

### BACKGROUND OF THE INVENTION

Current methods to control hemorrhage have limited effectiveness and may destroy healthy tissue. One method uses chemical or biological clotting enhancing agents or “glue.” When “glue” is used, both good and damaged tissues are glued, rendering surgical repairs daunting. Other approaches use heat applied by diathermy or ultrasound technologies. This approach stops the bleeding by burning the bleeding tissues and coagulating them.

The invention relates to treatment of wounds. In particular, it relates to a method and apparatus to reduce or stop bleeding in wounds where there may be a lack of medically-trained personnel, a lack of time, or a lack of medical equipment, such as in a combat environment or any other scene of injury. This invention is particularly suited to reduce or stop bleeding in wounds such as of the femoral artery and large vessels of the arms. Further, the invention may be beneficially adapted for use in intra-abdominal or intra-thoracic trauma.

### SUMMARY OF THE INVENTION

One embodiment of the invention provides an apparatus for inhibiting or stopping hemorrhaging of a wound. A casing includes a nozzle to be inserted into the wound. A torus-shaped flexible element is disposed in the casing and defines a longitudinal direction. An actuating device moves said flexible element in the longitudinal direction through the nozzle and into the wound. The approach allows the application of an internal tamponade structure to complement external pressure and the delivery of the treatment to the relevant site. In addition, the presence of electrode elements disposed on the surface of the torus-shaped flexible element permits electrical stimulation to cause vasoconstriction, thus limiting hemorrhage. In addition, pharmacological agents such as pro-coagulants present on the surface of torus-shaped flexible element permit the location of these substances at the site of maximum benefit.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a sectional view of one embodiment of a rolling tube apparatus of the invention.

FIG. 2 shows a perspective view of the torus-shaped flexible element of the apparatus of FIG. 1.

FIG. 3 shows a sectional view of the torus-shaped flexible element of FIG. 2.

FIG. 4 shows a longitudinal cross sectional view of the torus-shaped flexible element of FIG. 2.

### DETAILED DESCRIPTION OF THE INVENTION

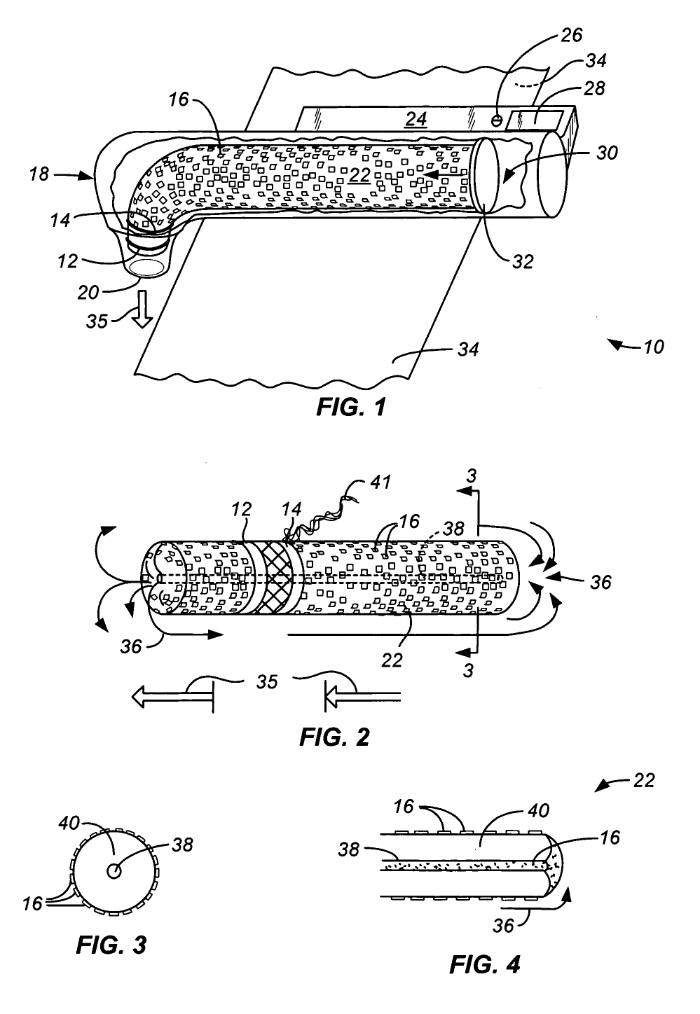

FIG. 1 illustrates an embodiment of a rolling tube apparatus 10 for treating a wound of a patient. The rolling tube apparatus 10 includes an adhesion ring 12, an electrode contact ring 14, an outer casing 18, a torus-shaped flexible element 22 disposed within casing 18, and treatment elements 16 disposed on element 22. In one embodiment, treatment elements 16 are electrodes, and element 22 and electrodes 16 are collectively called a donut or donut electrode. In this embodiment, no treatment elements exist between adhesion ring 12 and the electrode attachment ring 14.

The embodiment illustrated in FIG. 1 also includes a treatment element delivery system or actuating device that includes an electronics housing 24, a plunger 32, a control button 26 for activating the electrode treatment elements by the electronic stimulation and control circuitry and for releasing pressurized fluid from a chamber 28 into a space 30. Pressing the control button 26 of FIG. 1 releases pressure from pressure chamber 28 into space 30, moving plunger 32. The pressure causes the flexible element 22 to migrate toward an application nozzle 20 located at the distal end of casing 18. The adhesion ring 12 and the electrode attachment ring 14, or either ring individually, adhere to the internal surface of application nozzle 20. Therefore only the internal portion of element 22 rolls forward toward and through nozzle 20 to be extruded from the housing in a rolling manner as shown in FIG. 2. When nozzle 20 is placed against a wound in a patient, the rolling movement of element 22 is stopped by the torus-shaped flexible element reaching the full length of the wound track, whereby it can go no further because of tissue resistance. Plunger 32 prevents fluid in space 30 from gaining access to the patient's wound via central lumen 38 of torus-shaped flexible element 22. The added advantage of this process is that using this device and method, the torus-shaped flexible element follows the pre-existing wound track and creating new tracks can be avoided.

Pressing the control button 26 of FIG. 1 activates the electrode treatment elements 16 depicted in FIG. 1, FIG. 2, and FIG. 4 by means of the electronic stimulation and control circuitry contained within electronics unit 24 of FIG. 1. Activating the electrode treatment elements occurs via connecting wires 41 of FIG. 2 which are connected to the electrode attachment ring 14. Connecting wires 41 of FIG. 2 join electronics unit 24 of FIG. 1 to the electrode attachment ring 14. Connecting wires 41 of FIG. 2 may alternatively be made of conducting material or materials other than wires. Electrode treatment elements 16 are connected to the electrode attachment ring 14 of FIG. 2 with wires or other electrically conducting material or materials. Activating the electrode treatment elements creates a pulsed electrical signal in the electrode treatment elements similar to the painless injection system described in parts of U.S. application Ser. No. 10/746,685, filed Dec. 19, 2003, U.S. application Ser. No. 11/022,269, filed Dec. 22, 2004, and U.S. application Ser. No. 10/195,171, filed Jul. 16, 2002, which are incorporated herein by reference in their entirety. The torus-shaped treatment element unfurls into and along the wound track causing the electrode treatment elements to deliver an electrical signal that is an analgesic or anti-hemorrhagic, or both analgesic and anti-hemorrhagic, to the tissues that the electrode treatment elements come into contact with. The electronics is similar in action to that described in the painless injection system described in the U.S. Applications cited above. Each electrode treatment element is individually and separately activated, ensuring current flow to that element. This provides surety of current delivery along the whole surface of torus-shaped flexible element.

This system can be made to operate fully automatically, with the exception of the operator holding or securing the system nozzle 20 of FIG. 1 in the wound entry point while the device unfurls. The embodiment illustrated in FIG. 1 of rolling tube apparatus 10 further includes and adhesive strapping 34 for holding rolling tube apparatus 10 in place on a patient. The operator may secure the apparatus to the patient using the attendant adhesive strapping. Other retention methods known in the art alternatively may be used. This embodiment also includes a nozzle 20 for insertion into the wound opening and a torus-shaped flexible element 22. The approach allows the application of an internal tamponade structure to complement external pressure, the delivery of the electrode treatment elements to the relevant site, or the delivery of pharmacological agents, such as vaso-active substances, antibiotics, pro-coagulants, or other agents, or delivery of both electrode treatment elements and pharmacological agents such as vaso-active substances, antibiotics, pro-coagulants, or other agents.

The torus-shaped flexible element 22 may be formed of an elastic or other flexible material such as rubber, latex or other biologically acceptable pliant and elastic material. Torus-shaped flexible element 22 may be in the shape of an elongated torus or toroid. The torus-shaped flexible element 22 in this embodiment is a donut electrode. The torus-shaped flexible element 22 defines a longitudinal direction and includes an outer surface having a first area facing in a radially-inward direction, and a second area facing in a radially-outward direction. A plurality of treatment elements 16 is at least disposed on at least the first area of the outer surface of the torus-shaped flexible element 22. Each treatment element is activated individually. The actuating device moves the plunger 32 in a longitudinal direction. Thus, the torus-shaped flexible element 22 unfurls in a longitudinal direction through the nozzle 20 and through the wound opening in a direction indicated by arrow 35, into the wound. At least a portion of the first area faces in the radially outward direction and the treatment elements 16 on the portion of the first area engage a wall of the wound.

FIG. 2 illustrates a direction 36 of the unfurling direction of the torus-shaped flexible element 22. FIG. 2 illustrates a central lumen 38 of the torus-shaped flexible element 22. The torus-shaped flexible element 22 of the invention unfurls from its interior or luminal surface along the wound track in direction 36 of FIG. 4. Forward movement of the torus-shaped flexible element 22 is made from the central (orifice) area of the torus-shaped flexible element 22, with the outer edges of the torus-shaped flexible element 22 remaining fixed and in contact with the wound edges. The outer surface rolls forward as the torus-shaped flexible element's luminal surface migrates forward and stretches as it moves radially in an outward direction 36 of FIG. 2, eventually becoming the outer surface pressing against the wall of the wound. Because the unfurling is produced by the internal luminal surface of the torus-shaped flexible element 22, no sheering or longitudinal forces are produced, such as would occur if a probe, guide or standard catheter were used. This technique avoids any additional damage to the wound area. Further, because pressure is applied evenly across the wound surface by the fluid-filled torus-shaped flexible element, beneficial tamponade pressure at right angles to the wound surface is produced.

FIG. 3 illustrates a cross-sectional view of flexible element 22 along line 3-3 of FIG. 2. FIG. 3 illustrates a central lumen 38 and a fluid filled area 40 of flexible element 22. Internal pressure in space 40 maintains a degree of rigidity in the flexible element, but allows some flexibility of the flexible element.

FIG. 4 illustrates a longitudinal cross-sectional view of flexible element 22 along its center line. A central lumen 38 is shown along with a fluid filled area 40 of flexible element 22. Electrode elements 16 are disposed at least on the radially-inward facing outer surface of the flexible element 22. FIG. 4 also illustrates a direction 36 of an unfurling surface of flexible element 22.

In one embodiment of the invention, the treatment elements 16 may be electrode elements that are less than 4 square millimeters in area. In one embodiment of the invention, there may be about ten to hundreds of electrode treatment elements on the torus-shaped flexible element 22.

An advantage of the invention is that bleeding of a wound may be reduced or stopped by electrically stimulating the associated tissue using the electrode treatment elements. Low power electrical tissue stimulation is delivered via electrode treatment elements to cause vascular smooth muscle contraction, constricting the lumen of blood vessels, and limiting wound hemorrhage.

Low power electrical signal application to electrically excitable tissue such as nerve, muscle and smooth muscle causes the tissue to become activated. In the case of muscle, this results in the initiation of contraction. Applying a repetitive stimulus results in muscle tissue becoming, and remaining, contracted for the duration of signal application. Contraction of the smooth muscle layers in the wall of arterial vessels is the natural response of these tissues to injury, rupture, or breach, and has the potential to substantially reduce or stop blood loss from the vessel. Effectively applying an electrical signal to a heterogeneous tissue (from an electrical standpoint) requires specific features of the electrodes used and of the control unit supplying and distributing the signal to them. These attributes are adaptable to a wound dressing or internal self directed electrode probe array. In the operating theatre, during diathermy use, electrical stimulation to a bleeding vessel in muscle sometimes causes the bleeding vessel to retract. Using the invention, retraction of the bleeding vessel will not occur when a field of stimulation is used, since the associated areas will be stimulated evenly, rather than in one locality. As a consequence, the muscle as a whole, along with the bleeding vessel, will contract and will remain in contact with the electrode. The dressing and electrode of one embodiment of the invention are suitable for an open wound or gash. In another embodiment of the invention, the dressing and electrode are suitable for use where a track or tunnel type of wound exists in a patient, in which the bleeding vessel is away from surface access.

The rolling tube apparatus using electrode treatment elements may be adapted such that it is inserted to target different types of tissue. By way of example intra-abdominal or intra-thoracic bleeding may be reduced or stopped by stimulation of the sympathetic ganglion system or chain. In these circumstances, the introduction method of the device would be to locate the femoral artery (by Doppler ultrasound or other means), and breach the skin down to the fascia of the Psoas muscle. The rolling tube nozzle is then placed into the Psoas muscle which will guide the rolling tube, sub-facially, in a cranial direction towards the sympathetic ganglion chain, where electrical stimulation will beneficially cause reflex vasoconstriction in the associated vascular tree. The inherent nature of the rolling tube to follow the path of least resistance allows the electrodes to be located in the correct anatomical location by placing the electrode elements in close proximity to the sympathetic ganglion chain, located on the posterior abdominal wall.

When a heterogeneous tissue and its surface contact an electrode, several electrical signal routes are possible. The presence of “short circuit” electrical signal routes can reduce or eliminate current flow away from the target tissue, such as vascular smooth muscle, even though the electrode surface may have direct contact with the target tissue. By breaking the electrode up into multiple elements, in a tile-like fashion, the stimulant signal can be applied rapidly but separately in succession to each element, giving some surety of current delivery to the target tissue. By this arrangement, short circuiting through some areas is accommodated without causing failure of treatment across the entire stimulant area. The electronics system used herein has many qualitative similarities to that of U.S. application Ser. No. 10/746,685, U.S. application Ser. No. 10/022,269, and U.S. application Ser. No. 10/195,171, incorporated herein by reference in their entirety. This design element may be used for other applications, including a wound dressing which has analgesic properties.

The electrical generator unit and open wound electrode employ physically robust technology. The power levels required of the electrical generator for tissue stimulation are orders of magnitude below those that are required for diathermy and the electronics required for enabling the signal and distribution are within the parameters of relatively simple designs. The enabling electronics design and build features that have been disclosed in U.S. application Ser. No. 10/746,685, U.S. application Ser. No. 10/022,269, and U.S. application Ser. No. 10/195,171, incorporated herein by reference in their entirety, may be substantially the same as those that may be used in the invention for hemorrhage control. Differences may arise in the required pulse width (where muscle stimulation generally requires longer pulse widths compared to nerve), pulse frequency, and power, voltage, current flow settings and total number of electrode elements being controlled by switching circuitry. The direct tissue contact allows for significantly lower voltage settings than in the above-identified and incorporated patent applications. The invention may include a significantly greater number of electrode elements than disclosed in the above-identified patent applications, perhaps greater by one to three orders of magnitude. For example, the invention may include hundreds of electrode elements. Thus, the invention may include differences in the switching and control circuitry as well as the microprocessor unit and control program as compared to the above-identified patent applications.

The open wound dressing electrodes are made of a pliant material suitable for direct (internal) wound surface tissue exposure and capable of the electrical characteristics required for setting the electrode treatment elements in place.

In another embodiment of the invention, the treatment elements may also, or alternatively, be pharmacological agents.

Yet another embodiment of the invention provides a method for treating a wound of a patient. A torus-shaped flexible element is provided defining a longitudinal direction and including an outer surface having a first area facing in a radially-inward direction and a second area facing in a radially-outward direction. A plurality of treatment elements are provided on the first area of the outer surface of the flexible element. The flexible element is rolled in the longitudinal direction into the wound such that at least a portion of the first area faces in the radially outward direction and the treatment elements on the portion of the first area engage a wall of the wound. The treatment elements are controlled individually.

For the technically simpler case of an open wound, another embodiment provides an adapted standard dressing applied to the open wound surface. A typical wound dressing is usually made of cotton or other similar material. In one embodiment of the invention, the wound side of a wound dressing has an array of electrode elements, making contact with the wound surface. The electrode treatment elements are electrically networked with the signal generator unit. This embodiment may have tens to hundreds or more electrode treatment elements. The wound dressing may have several electronic components built into its substance. This reduces the number of individual wire leads emanating from the control unit. The dressing may also have various pharmacological agents incorporated into its substance (such as coagulation activators or antibiotics or both coagulation activators and antibiotics). Because of the nature of Trans Epithelial Nerve Stimulation (“TENS”) current, an additional benefit may be a degree of contact surface analgesia or anesthesia depending on various aspects of the signal applied.

Yet another embodiment of the invention provides using the self directing nature of the torus-shaped treating element, unfurling along the line of least resistance, to apply electrical stimuli to the sympathetic ganglion chain. Since the sympathetic ganglion chain controls, amongst other things, the blood flow to the intra-abdominal organs, this would reduce or stop hemorrhaging in an indirect manner, as contrasted with the electrical stimulation provided in other embodiments by direct application to the wound or hemorrhaging structure. The torus-shaped flexible element having disposed on it a plurality of electrode treatment elements could be introduced into an anatomically suitable location, for example within the fascial covering of the Psoas muscle or in the groin. The torus-shaped flexible element would unfurl and follow the anatomical outline, for example, of Psoas, towards Psoas's origin, locating the surface of the electrode elements in the region of the sympathetic ganglion chain on the posterior abdominal wall. This then allows the application of electrical stimuli to similar anatomical structures allowing for other local uses, for example, within the thorax. Thus, the electrode array can be located at a number of useful anatomical sites directly because of the nature of the torus-shaped flexible element and treatment elements.

An embodiment of the invention may be used for a bullet track or other track into a patient's body creating a wound where the source of hemorrhage may be within the track. In order to achieve hemorrhage control and wound stabilization, current ideal management requires wound exploration and tamponade. The invention provides a wound instrumentation system coupled with a surface electrode array and the incorporation of various pharmacological interventions. The projected ease of use may be sufficient to allow a minimally-trained or untrained personnel in the field to utilize the technique. A person may hold the nozzle of the applicator against the wound entry point, apply the adhesive strapping or other retention method associated with the unit or units, and press a button.

The invention proposed herein is works by enhancing the bodies own systems, causing vasoconstriction and thus, limiting blood flow and hemorrhage. It specifically does not destroy tissue. When the patient is in a place where surgical repair is possible to the damaged tissue, the device can be deactivated and removed. The system incorporates several basic routes to stem hemorrhage. First, local tamponade, especially in the bullet track model, is improved. Second, electrical stimulation of vascular smooth muscle maximizes active vessel occlusion. Third, the ability to deliver pharmacological vaso-active agents or other agents, such as antibiotics, pro-coagulants, etc., to the site (especially in the case of the bullet track model) allows biochemical processes to complement the physical forces producing hemorrhage control. These combine in a reversible manner, such that optimal stabilization may be produced in the field and during transit of the patient to a suitable surgical unit. Using the invention disclosed herein, rapid hemorrhage control and some local analgesia will be effectively delivered, with no further tissue damage beyond the initial injury. This will allow the casualty to be stabilized and transferred to a suitable surgical site with the potential for reparative surgery maximized. This allows for the best outcome for the patient, by stabilizing the blood loss in the acute situation, and allowing transfer to a proper place for medical/surgical care without causing additional destruction to the tissues. This gives the surgical team the best range of options for repairing damaged tissues and structures.

The invention may be especially applicable to limb wounds. Intra-thoracic or abdominal wounds may also be treated with the invention particularly in cases where there is a clear wound track that the tube electrode could follow to the hemorrhagic tissue. Where the technique is to be applied for intra-thoracic or intra-abdominal hemorrhage control, access to other tissue (such as the sympathetic ganglion chain) may be facilitated by alternate insertion designs or devices. These capitalize on the self directing (blunt dissection) capabilities of the rolling tube design. The invention may also be used in a hollow viscous or may be used in causing the viscous to contract and create pressure on a bleeding surface (e.g. incorporation of the technology into a Sengstaken tube for use in upper GI hemorrhage).

Other uses of the apparatus include instrumentation of the vascular tree, particularly where there is damage to the blood vessel. The general features of the design also render it appropriate for uses such as traumatic or degenerative rupture of the aorta. These uses are possible because of the self directing nature of the rolling tube structure.

Another use of the apparatus is instrumentation of tendon sheaths, joints and other synovial spaces. This use is possible because of the self directing nature of the rolling tube structure.

On one embodiment, the generator unit may have a power output of 10 watts or less, and in another embodiment has a power output of 5 watts or less.

In one embodiment, the generator unit may have dimensions of 4 centimeters by 10 centimeters by 19 centimeters or less.

The open wound dressing may be of standard dressing pack size(s). The bullet track unit may be similar in size to the generator.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An apparatus for inhibiting hemorrhaging of a wound of a patient, said apparatus comprising:
a casing including a nozzle configured to be inserted into the wound;
a torus-shaped flexible element disposed in the casing and defining a longitudinal direction; and
an actuating device configured to move said flexible element in the longitudinal direction through said nozzle and into the wound.

2. The apparatus of claim 1 wherein said actuating device comprises a plunger.

3. The apparatus of claim 1 further comprising an adhesion ring configured to limit the movement of said flexible element.

4. The apparatus of claim 1 further comprising an adhesive strapping configured to retain said casing on the patient.

5. An apparatus for treating a wound of a patient, said apparatus comprising:
a torus-shaped flexible element defining a longitudinal direction and including an outer surface having a first area facing in a radially-inward direction and a second area facing in a radially-outward direction;
a plurality of treatment elements disposed on said first area of said outer surface of said flexible element; and
an actuating device configured to roll said flexible element in the longitudinal direction into the wound such that at least a portion of said first area faces in the radially outward direction and said treatment elements on the portion of the first area engage a wall of the wound.

6. The apparatus of claim 5 wherein said actuating device comprises a plunger.

7. The apparatus of claim 5 further comprising an adhesion ring configured to limit the movement of said flexible element.

8. The apparatus of claim 5 further comprising a casing including a nozzle configured to be inserted into the wound, said flexible element being disposed in said casing.

9. The apparatus of claim 8 further comprising an adhesive strapping configured to retain said casing on the patient.

10. The apparatus of claim 5 wherein said treatment elements comprise at least one of pharmacological agents and electrode elements.

11. The apparatus of claim 10 wherein said treatment elements comprise electrode elements, said apparatus further comprising electronics configured to source electrical current to said electrode elements.

12. The apparatus of claim 11 wherein the electrical current comprises TENS current.

13. A method for treating a wound of a patient, comprising the steps of:
providing a torus-shaped flexible element defining a longitudinal direction and including an outer surface having a first area facing in a radially-inward direction and a second area facing in a radially-outward direction;
providing a plurality of treatment elements on at least said first area of said outer surface of said flexible element; and
rolling said flexible element in the longitudinal direction into the wound such that at least a portion of said first area faces in the radially outward direction and said treatment elements on the portion of said first area engage a wall of the wound.

14. The method of claim 13 wherein said rolling step includes using a plunger to actuate said flexible element.

15. The method of claim 13 comprising the further step of limiting the movement of said flexible element by use of an adhesion ring.

16. The method of claim 13 comprising the further steps of: providing a casing including a nozzle, said flexible element being disposed in said casing; and
inserting said nozzle into the wound before said rolling step.

17. The method of claim 16 comprising the further step of using an adhesive strapping to retain said casing on the patient.

18. The method of claim 13 wherein said treatment elements comprise at least one of pharmacological agents and electrode elements.

19. The method of claim 18 wherein said treatment elements comprise electrode elements, said method comprising the further step of sourcing electrical current to said electrode elements.

20. The method of claim 19 wherein the electrical current comprises TENS current.
